(19) United States
(12) Patent Application Publication
(10) Pub. No.: US 2008/0275355 A1
(43) Pub. Date: Nov. 6, 2008

(54) Method For Diagnosing An Infectioin Condition

(75) Inventors: Khosrow Namjou-Khaless, Norman, OK (US); Gina Lynn McMillen, Norman, OK (US); Chad Barrett Roller, Oklahoma, OK (US)

Correspondence Address: TOMLINSON & O'CONNELL, P.C., TWO LEADERSHIP SQUARE, 211 NORTH ROBINSON, SUITE 450, OKLAHOMA CITY, OK, 73102, US

(73) Assignee: Ekips Technologies, Inc., Norman, OK (US)

(21) Appl. No.: 11/744,601

(22) Filed: May 4, 2007

(51) Int. Cl. A61B 5/00; G01N 21/00; G01N 33/497

(52) U.S. Cl. 600/532; 356/300; 73/23.2; 73/23.3

(57) ABSTRACT

A method and an apparatus for determining an infection condition in an organism by measuring the level of nitrous oxide present in a gas sample taken from the organism. In one embodiment the nitrous oxide content of a gas sample is measured to diagnose systemic inflammatory response in a living organism. In an alternative embodiment, the nitrous oxide level of a gas sample taken from a living organism may be compared the with an expected nitrous oxide level for a healthy organism or with a prior measured nitrous oxide level of the living organism to diagnose the presence or absence of an infection in the living organism. A method and apparatus for determining response to a course of therapy is provided. The method and apparatus compares the nitrous oxide levels of a living organism before and after the administration of a therapy to the living organism to determine a response to the therapy.

### FIELD OF THE INVENTION

The present invention is generally directed to the field of diagnosing the presence of an infection condition or infectious disease based upon the presence of a biomarker in a gas sample and more specifically to the diagnosis of sepsis-like inflammatory response using a concentration of endogenous nitrous oxide (N₂O) in a gas sample from a living organism.

### SUMMARY OF THE INVENTION

The present invention is directed to a method for diagnosing systemic inflammatory response in a living organism. The method comprises collecting a first gas sample from the living organism and measuring the nitrous oxide content of the first gas sample to acquire a first measured nitrous oxide value. The first measured nitrous oxide value is then compared to a nitrous oxide reference value typical for a healthy and similar living organism to determine the presence of systemic inflammatory response.

The present invention also includes a method for diagnosing the presence or absence of an infection in a living organism. The method comprises obtaining a first gas sample from the living organism and measuring at least one biomarker level in the first gas sample to obtain a measured biomarker level. The at least one biomarker may comprise nitrous oxide and the measurement may obtain a measured nitrous oxide level. The method further includes comparing the measured nitrous oxide level with an expected nitrous oxide level for a healthy organism or with a prior measured nitrous oxide level in the living organism and diagnosing the presence or absence of the infection condition based on the comparison.

The present invention further includes a method for diagnosing sepsis in humans. The method comprises collecting a first gas sample from a living organism and measuring a biomarker level present in the first gas sample to acquire a first measured biomarker value. The present method also includes comparing the first measured biomarker value to biomarker levels for either a living organism not having sepsis or for the same living organism at an earlier time to diagnose the presence or absence of sepsis.

Further still, the present invention is directed to a method of diagnosing systemic inflammatory response in a human comprising detecting a level of endogenous nitrous oxide in at least one sample of expired air taken from said human and diagnosing whether said human has systemic inflammatory response based on said level of endogenous nitrous oxide.

Still yet, the present invention includes a system for the analysis of a breath sample. The system comprises a means for accepting a gas sample from a living subject, a means for measuring an amount of endogenous nitrous oxide present in the gas sample, and a means for analyzing the level of endogenous nitrous oxide in the gas sample to determine the presence or absence of systemic inflammatory response.

The present invention further includes a method for detecting response to therapy in a living organism. The method comprises collecting a first gas sample from the living organism and measuring a nitrous oxide level of the first gas sample to acquire a first measured nitrous oxide value. Next, a therapy is administered to the living organism and a second gas sample is collected. A nitrous oxide level of the second gas sample is measured to acquire a second measured nitrous oxide value and compared to the first measured nitrous oxide value to determine a response to the therapy.

Additionally, the present invention is directed to a method for discovering a drug therapy for a living organism. The method comprises collecting a first gas sample from the living organism and measuring the nitrous oxide level of the first gas sample to acquire a first measured nitrous oxide value. A therapy is administered to the living organism and a second gas sample is collected from the living organism. The nitrous oxide level of the second gas sample is measured to acquire a second measured nitrous oxide value and then compared to the first measured nitrous oxide value to determine an effectiveness of the drug therapy.

### DESCRIPTION OF THE FIGURES

FIG. 1 is a diagrammatic representation of a system used to collect and measure biomarker levels in a gas sample.

FIG. 2 is an alternative embodiment of a system used to collect and measure biomarker levels in a gas sample.

FIG. 3 is a graph showing measured biomarker levels from gas samples collected from a bovine test subject. The graph shows nitrous oxide and carbon dioxide signal amplitudes in exhaled breath from a bovine subject as measured by the system of FIG. 1.

FIG. 4 is a graph showing exhaled nitrous oxide concentration levels in a bovine test subject at 24 hours and 72 hours post-infection. The nitrous oxide concentration levels are shown normalized to carbon dioxide concentrations in a bovine test subject challenged with bacteria.

FIG. 5 is a graph of measured concentration of exhaled carbon dioxide and exhaled nitrous oxide over an exhalation period.

FIG. 6 is a bar graph illustrating the ratio of nitrous oxide concentration to carbon dioxide concentration in asthmatic subjects versus non-asthmatic subjects.

FIG. 7 is a bar graph illustrating measured nitrous oxide to carbon dioxide ratios for a subject over a three day test period. The subject experienced symptoms of infection on day one and no symptoms of infection on days two and three.

FIG. 8 is a bar graph illustrating the measured nitrous oxide to carbon dioxide ratios in a subject over a nine day period. The subject exhibited symptoms of acute infection on days one through three and no symptoms on days seven through nine.

FIG. 9 is a line graph showing respiration rate measured over the study period for infected primates and a zero dose control primate.

FIG. 10 is a line graph showing end-tidal carbon dioxide concentrations for primate subjects infused with bacterium versus an uninfected primate subject.

FIG. 11 is a line graph showing heart rate measurements for primate subjects infused with bacterium versus an uninfected primate subject.

FIG. 12 is a bar graph showing the absolute exhaled nitrous oxide to carbon dioxide ratios for primate subjects infused with E. coli or B. anthracis compared to an uninfected primate subject.

FIG. 13 is a bar graph showing the nitrous oxide concentration measurements of a primate test subject infused with E. coli.

FIG. 14 is a bar graph showing an average change in the ratio of exhaled nitrous oxide to carbon dioxide for two E. coli and one anthrax challenged primate subjects and one control or zero bacterial dose subject.

### DESCRIPTION OF THE INVENTION

The measurement of biomarkers in a gas sample has proven to be an efficient way to detect the presence or absence of a wide variety of biological conditions. For example, the measurement of NO in exhaled breath has been found to give an indication of lower airway inflammation without requiring the use of other more costly tests, such as lung biopsies. One such method and system for assessing pulmonary function using laser spectrometry is disclosed in U.S. Pat. No. 7,192,782 issued to Ekips Technologies, Inc., the contents of which are incorporated herein by reference. Biomarker molecules have been discovered as indicators of various biological conditions such as diabetes, cancer, cystic fibrosis, oxidative stress, and infectious disease. However, there remains a need for the development of new methods and systems used to measure biomarkers present in a gas sample to assist care givers in treating an infected living organism. In the case of the present invention nitrous oxide or an isotope thereof may be used as a biomarker indicative of the presence or absence of an infection condition in a living organism.

In the United States, sepsis is a leading cause of death in non-coronary intensive care unit patients and is a major cause of death in intensive care units worldwide. The mortality rates for individuals with sepsis are approximately 20% and 40% for severe sepsis and 60% for those with septic shock. The symptoms of sepsis are often related to an underlying infection condition. In sepsis and sepsis like conditions, the immune system of the organism misdirects the reaction of the body find the physiological process of inflammation spirals out of control and threatens the health and life of the organism within a few hours. Accordingly, there is an ongoing need to develop rapid methods to assess the severity of infection and the status of immune system response resulting in sepsis.

Currently infection conditions are diagnosed through the use of blood tests as well as symptom based diagnosis using criteria such as body temperature, heart rate, and respiratory rate. Though there are excellent drugs and therapies currently available to treat infection, the inability to diagnose an infection condition rapidly before the symptoms and infection have progressed to a severe state limits the effectiveness of such treatments and leads to the high mortality rates discussed herein. Accordingly, there is a high time value proposition in the ability to recognize and treat infection prior to the onset of sepsis.

In the present invention, the inventors have developed a new method and system for measuring the presence and the progression of infection. They have determined there is a direct relationship between exhaled breath levels of a biomarker and infection in a living organism. In a preferred embodiment, measured levels of nitrous oxide have been shown to indicate the presence of an infection condition in a test subject. Specifically, elevated nitrous oxide levels are shown herein as an indicator of Bovine-Respiratory Disease, an acute infection in humans, and immune system response in primates.

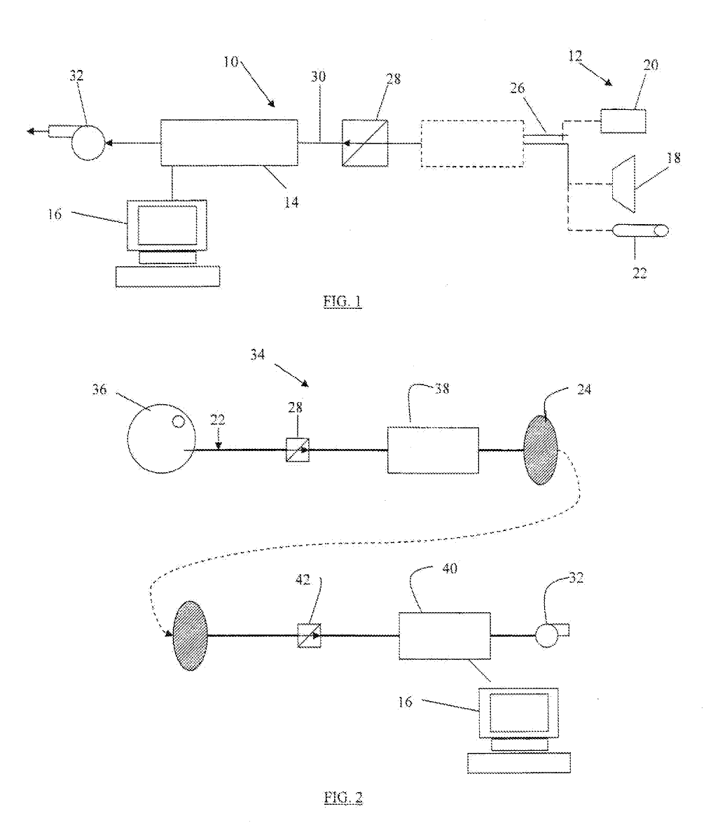

Turning now to the figure and in particular to FIG. 1, there is shown, therein an exemplary system 10 used in the analysis of a gas sample. The system 10 may comprise a means for accepting the gas sample 12 from a living subject, a means for measuring an amount of a biomarker in the gas sample 14, and a means for analyzing the level of biomarker 16 in the gas sample to determine the presence or absence of systemic inflammatory response.

The means for accepting the gas sample 12 may comprise any device used to collect a gas sample from a living organism. Such means may include a face mask 18 adapted to cover the nose and mouth of the living organism, a nasal canula (not shown), or a mouthpiece 20 adapted to be held in the subject's mouth during exhalation. Alternatively, the means may comprise a ventilator and intubation tube 22 connected to a gas sample bag 24 (FIG. 2). For purposes of illustration the system of FIG. 1 will be discussed with reference to the mouthpiece 20.

The mouthpiece may be connected to a T-shaped junction (not shown) that is configured to carry a portion of the gas sample away to a discard bag (not shown). If so equipped, the T-junction may have a one-way valve constructed to prevent air passing into the discard bag from reentering the tube 26. Otherwise, the mouthpiece is connected directly to the measuring means 14 via a flow controller 28 adapted to regulate the flow of the gas sample into the measuring means. The flow controller 28 may also have a one-way valve (not shown) designed to prevent the flow of air towards the mouthpiece 20. The flow controller 28 may be connected to the means for measuring biomarker levels 14 using commercially available tubing 30 appropriate for such applications.

The means for measuring the levels of biomarker 14 present in the gas sample may comprise a means for measuring an amount of endogenous nitrous oxide present in the gas sample. Such means may comprise any device used to measure trace gases present in a gas sample. Accordingly, the means may comprise an electrochemical cell or a spectrometer gas sample cell, which can be a Herriott cell or multipass White cell. Additionally, the device may comprise a sensor adapted to measure the concentration of a reference gas present in the gas sample. Such reference gases may include water vapor (H₂O) or carbon dioxide. Further, the device may be integrated with existing equipment such as a ventilator or respiration meter without departing from the spirit of the invention.

By way of example only, an acceptable laser spectrometer system may comprise a mid-infrared tunable diode laser absorption spectroscopy system where the light source used to illuminate the gas sample comprises an IV-VI diode laser with an emission wavelength in the range of from about 3 μm to about 10 lira. The IV-VI diode laser may be controlled by a current driver/function generator assembly and a personal computer 16. It will be appreciated, however, that the means for measuring the endogenous nitrous oxide content of the gas sample may comprise any other system adapted to measure the level of trace gases present in a gas sample including, but not limited to, a chemiluminescence analyzer, a mass spectrometer, and a gas chromatography system.

Returning now to FIG. 1, a mechanical pump 32 may be used to evacuate the means for measuring the nitrous oxide content of the gas sample 14 and to keep the system 10 at a selected pressure. Mechanical vacuum pump 32 can be operated to produce a vacuum in the range of from approximately 10 Torr to approximately 80 Torr. The resulting flow rate can be in the range of from approximately 0.5 liters per minute to approximately 30 liters per minute. Further, pump 32 provides a pull on the system such that the subject is not required to overly exert itself when exhaling or otherwise providing the gas sample to system 10.

Turning now to FIG. 2 there is shown therein an alternative system 34 used to measure biomarker levels in an exhaled gas sample. The biomarker measuring system 34 of FIG. 2 is particularly useful in situations where the subject 36 is breathing with the aid of a ventilator (not shown) or is otherwise intubated 22.

The system 34 may comprise the intubation tube 22 and flow controller 28, discussed above, operatively connected to a carbon dioxide sensor 38 and the sample bag 24. The carbon dioxide sensor 38 and sample bag 24 may be in fluid communication with a pump (FIG. 1) adapted to draw exhaled air from the subject at a rate of 200 cc per minute through the carbon dioxide sensor 38 to measure the level of exhaled carbon dioxide in the gas sample and to ultimately pull the gas sample into the sample bag 24.

The gas sample, stored in sample bag 24, may then be taken to a device 40 used to measure the biomarker levels present in the gas sample. For example, the gas sample may be drawn into a laser spectrometer 40 through a flow controller 42 using a mechanical pump 32. The flow controller 42 is used to regulate the flow of the gas sample into the laser spectrometer 40. For example, the flow controller 42 may limit the flow rate into the laser spectrometer to 1.0 liter per minute.

Once the gas sample passes through the flow controller 42 it is drawn into the laser spectrometer 40 under the pull of the mechanical pump 32 where the selected biomarker(s) content of the gas sample is measured. In the embodiment shown in FIG. 2, the gas sample may be illuminated with a light beam from a laser diode (not shown) to detect the levels of nitrous oxide and carbon dioxide present in the gas sample. The light beam passes through the gas sample and impinges upon a light detector (not shown). The light detector detects the intensity of the light beam based on the presence or absence of a particular biomarker gas and provides an output voltage to computer 16 adapted to translate the output voltage into a measured biomarker value. In the case of a laser spectrometer as described herein, the laser beam is focused onto a mercury-cadmium-telluride detector positioned outside the Herriott cell using an aspheric ZnSe lens.

In an alternative arrangement the laser spectrometer may be adapted to measure the level of the biomarker present in the gas sample and the level of a reference gas, such as carbon dioxide, also present in the gas sample. The computer may then ratio the two values to provide an indication of increased or decreased biomarker levels relative to a known reference gas value. A preferred laser spectrometer system designed to measure a trace gas biomarker and a reference gas in exhaled breath is disclosed in U.S. Pat. No. 7,192,782.

The systems described herein may be used in a method for diagnosing systemic inflammatory response in a living organism. The method comprises collecting a first gas sample from the living organism using any one of the collection means described with reference to FIGS. 1 and 2. The gas sample may be orally exhaled breath, nasally exhaled breath, or a combination of both orally and nasally exhaled breath.

The gas sample flows from the collection means into a device used to measure the level of nitrous oxide present in the exhaled breath sample. Measuring the nitrous oxide level may be accomplished by illuminating the gas sample using a light beam from a spectrometer light source such as a diode laser adapted to illuminate the gas sample with infrared light. The laser spectrometer system measures the level of nitrous oxide by detecting the level of light absorption by the molecule of interest.

This measured value for exhaled nitrous oxide may then be compared to an exhaled nitrous oxide value for a healthy living organism to determine the presence of systemic inflammatory response based upon increased levels of nitrous oxide in the gas sample. Alternatively, the method may also comprise collecting subsequent gas samples from the living organism and measuring the nitrous oxide content of the subsequently collected gas samples to acquire nitrous oxide values. The later acquired nitrous oxide values may then be compared to the first measured nitrous oxide value to determine the presence of systemic inflammatory response.

The collection and measurement methods and systems disclosed herein may also be used in the development of new therapies for the treatment of infection conditions or alternatively for monitoring the effectiveness of treatments. For example, the effectiveness of a course of therapy may be monitored using the systems of the present invention by measuring the level of nitrous oxide in a first gas sample, administering the therapy, and collecting a second gas sample. The nitrous oxide levels of the first and second gas samples are compared to each other to determine tire effectiveness of the therapy, for example, a reduction in the concentration of nitrous oxide between the first and second gas samples may indicate the inhibition of the infection condition. The comparison of nitrous oxide levels may also lead to a determination as to the effectiveness of a medication dosage and adjustments made to the dosage as the nitrous oxide levels of further gas samples are measured. One such medication used to treat an infection condition in a living organism is Xigris™ manufactured by Eli Lilly Co.

In accordance with the present method, the gas sample measurement system may be adapted to also measure the concentration of a reference gas present in the gas sample. With reference to the systems described herein, the laser spectrometer may measure the concentration of the reference gas or a separate gas sensor may be used. For example, where exhaled carbon dioxide is used as the reference gas, a separate carbon dioxide sensor may be used to determine the concentration of exhaled carbon dioxide in the gas sample. This resulting value may then be used to determine a ratio of nitrous oxide to carbon dioxide content to determine a normalized measured nitrous oxide value. Such measured value may then be compared to a standard nitrous oxide value for a healthy individual to determine the presence or absence of systemic inflammatory response.

### Bovine Biomarkers

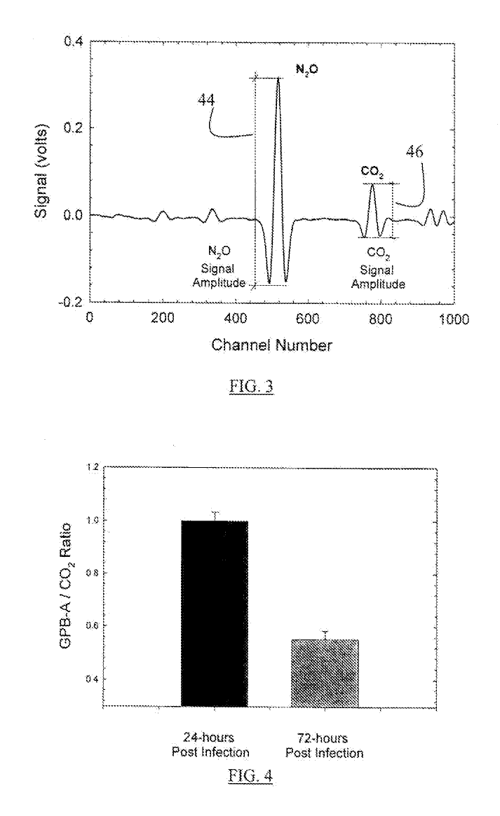

Turning now to FIG. 3 there is shown a graph of measured biomarker and reference gas in an exhaled breath sample of a living organism measured using the laser spectrometer system described herein. In the graph of FIG. 3 the measured biomarker comprises exhaled endogenous nitrous oxide and the reference gas comprises exhaled carbon dioxide. The graph shows the concentration of nitrous oxide and carbon dioxide as a voltage value based on the amplitude 44 for nitrous oxide and amplitude 46 for carbon dioxide. The graph of FIG. 3 shows the results of a measured gas sample from a steer infected with bacteria. The gas sample was measured using the system 10 of FIG. 1 with a facemask 18 having a non-rebreathing valve (not shown). The steer was challenged with pathogen in the form of Mannheimia haemolytica and Bovine Viral Diarrhea Virus (BVDV).

To record the data presented herein two measurements were performed on one steer using the method described herein and the apparatus shown in FIG. 1. The first measurement was taken 24-hours post challenge with pathogen and the second 72-hours post challenge. The gas sample comprised the steer's exhaled breath. The exhaled breath was collected by drawing breath from the steer 18 using a mask placed over the steer's muzzle and a pump 32, which sampled air at a flow rate of three (3) liters per minute.

The nitrous oxide concentration signal was normalized using exhaled carbon dioxide concentrations. As shown in FIG. 4, nitrous oxide levels dramatically decreased from the 24-hours post challenge measurement to the 72-hours post challenge measurement and correlated with a decrease in body temperature, a known parameter of the health of steers. Accordingly, the increased levels of nitrous oxide in the breath sample taken at 24-hours post challenge corresponds to the time at which the steer was exhibiting signs of infection, elevated body temperature, thus indicating that nitrous oxide is an exhaled biomarker indicative of an infection condition in cattle.

### Respiratory Condition Detection Example

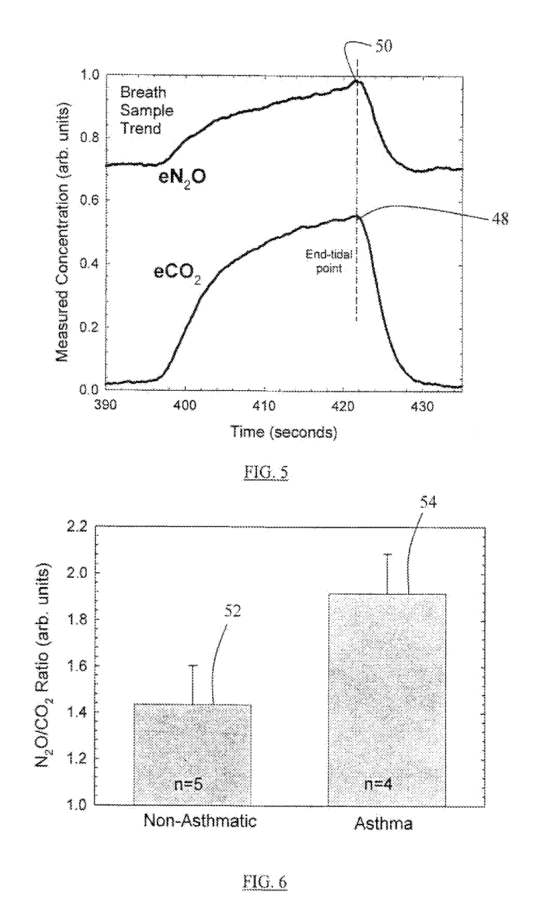

With reference now to FIGS. 5 and 6, the measurement of biomarkers to detect the presence or absence of a respiratory condition in humans will be discussed. Nitric oxide (NO) is a known potential biomarker used in the diagnosis of an asthma condition in humans. Previously mentioned U.S. Pat. No. 7,192,782 issued to Ekips Technologies, Inc. discloses the usefulness of measuring nitric oxide levels as they relate to a reference gas in human subjects. However, there remains a need for improved biomarkers that provide an indication of asthma in humans.

Accordingly, the present invention provides a method for determining the presence or absence of an asthma condition based upon the level of endogenous nitrous oxide (N₂O) present in an exhaled breath sample. In accordance with the present invention, breath samples of twenty-two subjects were analyzed using the system of FIG. 1 comprising a laser spectrometer to measure the level of endogenous nitrous oxide present in each gas sample.

The test subjects rinsed their mouths thoroughly with water before exhaling a single breath continuously into a mouthpiece for a period of approximately fifteen (15) seconds at a flow rate of three (3) liters per minute. The exhaled gas samples were analyzed with the laser spectrometer system to determine a measured concentration of exhaled nitrous oxide and a measured concentration of carbon dioxide over the entire exhalation time period.

As shown in FIG. 5, the levels of exhaled carbon dioxide and nitrous oxide increase over the exhalation phase until such point 48 as the level of exhaled carbon dioxide decreases dramatically. Immediately prior to this drop-off point is the end-tidal point of the exhalation phase. The nitrous oxide level corresponding to the end-tidal point 50 of the corresponding exhaled carbon dioxide level is taken and used to determine a ratio between the measured end-tidal nitrous oxide value and the measured end-tidal carbon dioxide value. This ratio may then be compared to the value of the same individual at a time when they were not suffering from the symptoms of a respiratory condition or to a value determined to be that of a person having normal lung function to determine the presence or absence of asthma or another respiratory condition.

FIG. 6 is a graph showing the average ratio of nitrous oxide relative to carbon dioxide from individuals suspected of being non-asthmatic 52 versus subjects strongly suspected of suffering from asthma 54. The graph shows a strong correlation between asthma and elevated nitrous oxide levels. Accordingly, nitrous oxide in an exhaled gas sample appears to provide an indication of asthma which may result from the presence of an infection condition in the lungs.

### Biomarker for Infection Condition Example

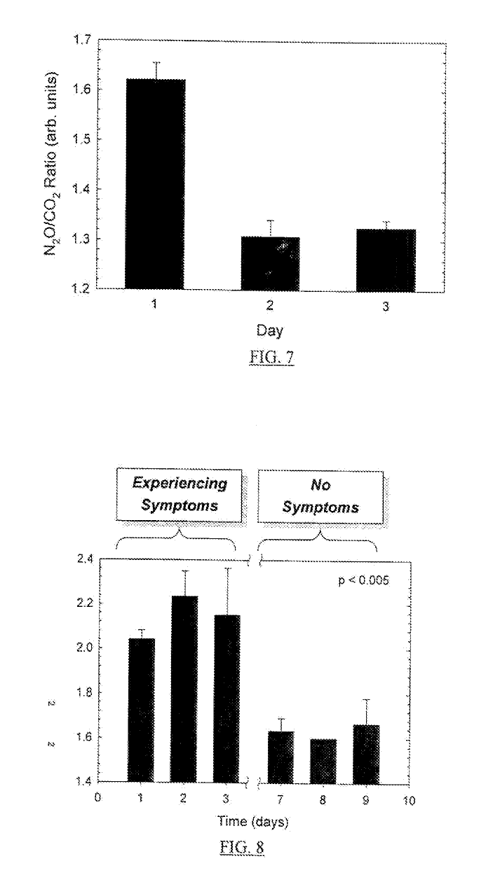

Turning now to FIG. 7, measured nitrous oxide values for a subject suffering from acute illness due to infection are shown therein. The subject was tested over three days and exhibited symptoms of infection in the form of elevated body temperature on day one and no observable symptoms on days two and three. The subject was tested using the system shown in FIG. 1 over three days and exhibited elevated nitrous oxide levels on day one and reduced nitrous oxide levels on days two and three.

FIG. 8 shows results from a series of exhaled breath tests taken over a nine day period. The subject was experiencing symptoms of acute illness in the form of elevated body temperature, nausea, and headache during the first three days of the sample period and no symptoms over the final three days of the sample period. The subject's exhaled breath samples were tested using the system disclosed with reference to FIG. 1 to determine the level of endogenous nitrous oxide exhaled over an exhalation period. As illustrated in the graph of FIG. 8, the subject exhibited elevated nitrous oxide levels in its exhaled breath when suffering from symptoms of fever, nausea, and headaches and lower nitrous oxide levels in the absence of fever, nausea, and headaches. Based on these results, the elevated levels of nitrous oxide in the subject's exhaled breath were determined to correlate to the presence of infection in the subject.

### Primate Biomarker Example

The following example discusses use of the present invention to detect the presence of an infection condition in a living organism comprising a primate. More specifically, the following set forth procedures and data used to determine the levels of nitrous oxide in a non-human primate model wherein the primates were challenged with either E. coli or Bacillus anthracis. The non-human primates used in the following study comprised three baboons. Two of the baboons were infected with E. coli while the third was infected with Bacillus anthracis.

The E. coli infected subjects were infused with E. coli over a period of two (2) hours. The infusion resulted in the development of infection type symptoms including a significant change in respiration rate, white blood cell counts, and body temperature and increased nitrous oxide levels in the subjects' exhaled breath. The increased nitrous oxide levels in the E. coli infected subjects may be due to E. coli's defense against immune system responses by denitrification, the conversion of anti-microbial nitric oxide to nitrous oxide or as a downstream product, possibly of nitroxyl (HNO), of the inflammatory immune system response.

Measurement of the E. coli infected subjects began at hour zero of tire study and continued until hour 8. Each subject was successfully infected as indicated by reduction in white blood cell counts, increased body temperature, and increased respiration rates.

Gas samples were collected from the subjects using the system 34 disclosed with reference to FIG. 2. Accordingly, the gas samples were collected from the subjects via an intubation tube 22 (FIG. 2) at a rate of 200 ml per minute. The gas sample was first directed to a carbon dioxide sensor 38 adapted to measure the concentration of carbon dioxide present in the exhaled breath sample as well as the respiration rate of the subject prior to being directed to a breath sample collection bag.

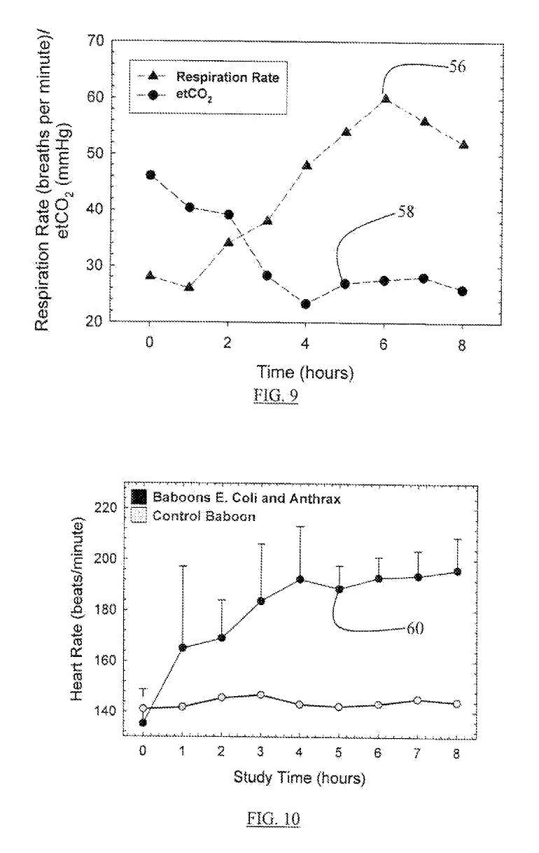

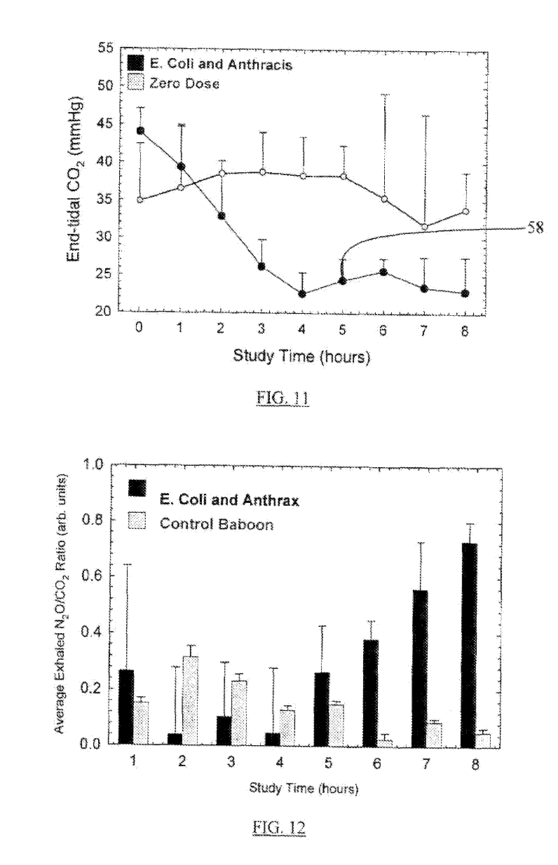

A portion of the subjects' exhaled breath was then directed to a previously described laser spectrometer sensor system for measurement of the endogenous levels of exhaled nitrous oxide and exhaled carbon dioxide present in the subjects' breath during the onset of infection. As shown in FIGS. 9, 10, and 11, as the infection progressed in time mid effect on the subjects, the subjects' respiration rate 56 and heart rate 60 (versus an uninfected subject) saw a marked increase. While the concentration of end-tidal carbon dioxide 58 (versus an uninfected subject in FIG. 11) decreased over the course of infection. The decrease in carbon dioxide levels is the result of the increased respiration rate and shallow breathing. Accordingly, the measured nitrous oxide values are normalized to the measured carbon dioxide values using the following equation.

$$\frac{{C}_{e {N}_{2} O}}{{C}_{e {CO}_{2}}} = \frac{{C}_{{N}_{2} O} - {C}_{a {N}_{2} O}}{{C}_{e {CO}_{2}}} ∝ \frac{{V}_{{N}_{2} O} - {V}_{a {N}_{2} O}}{{V}_{e {CO}_{2}}} \qquad EQ ( 1 )$$

The concentration of exhaled nitrous oxide is normalized to exhaled carbon dioxide because both molecules originate from the blood stream, diffuse across the pulmonary membrane, and are expired through the lungs. The result of normalization is the ratio of exhaled nitrous oxide to carbon dioxide. The concentration of endogenously produced nitrous oxide in exhaled breath (C_{eN}_{₂}_{O}) is determined by subtracting measured nitrous oxide in breath (C_{eN}_{₂}_{O}) from the ambient concentration of nitrous oxide (C_{aN}_{₂}_{O}). There is no need to subtract ambient carbon dioxide from exhaled carbon dioxide because the levels of ambient carbon dioxide are negligible.

FIG. 12 shows a graph of exhaled nitrous oxide to carbon dioxide ratios for an E. coli infected primate test subject and a B. anthracis infected test subject versus an uninfected control primate test subject. FIG. 12 shows the infected test subjects exhibited an increase in average exhaled nitrous oxide/carbon dioxide ratio (black bar) versus the nitrous oxide/carbon dioxide ratio (gray) of the control subject. The increased values shown in FIG. 12 corresponded to the onset of symptoms of infection including a significant change in respiratory rate, body temperature, and heart rate.

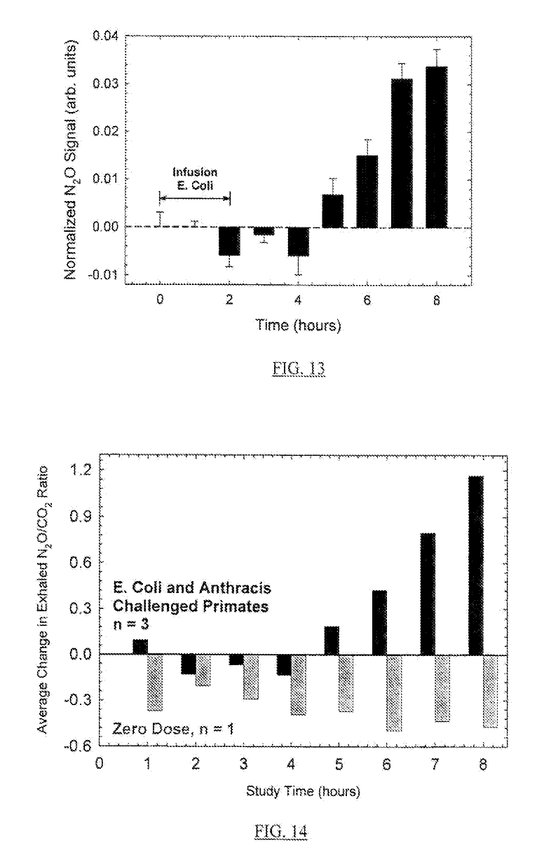

FIG. 13 shows a graph of nitrous oxide relative concentrations for a primate test subject infected with E. coli. The nitrous oxide measurements have been normalized to the concentration of carbon dioxide. As shown in FIG. 13, there was an initial decrease in nitrous oxide levels post infusion of bacteria followed by a dramatic increase in nitrous oxide levels from hour 4 to hour 8. The rise in exhaled nitrous oxide corresponded to the onset of symptoms of infection including increased respiratory rate, increased body temperature, and increased heart rate. This graph illustrates that an increase in exhaled nitrous oxide levels in the primate subject correlates to progression of the infection condition to sepsis.

FIG. 14 shows a bar graph to illustrate the average change in exhaled nitrous oxide to carbon dioxide ratios in infection challenged primates as discussed above. The bar graph of FIG. 14 plots the average change from baseline at hour 0 in nitrous oxide/carbon dioxide ratios of E. Coli and B. anthracis infected test subjects (black bar) versus nitrous oxide/carbon dioxide ratios for an uninfected test subject (gray bar). As shown in FIG. 14 the average change in exhaled nitrous oxide/carbon dioxide ratios for the infected subject (black bar) increased from below a baseline value of 0.0 to approximately 1.2 as the infection condition progressed from hour zero to hour eight.

Various modifications can be made in the design and operation of the present invention without departing from the spirit thereof. Thus, while the principal preferred construction and modes of operation of the invention have been explained in what is now considered to represent its best embodiments, which have been illustrated and described, it should be understood that within the scope of the appended claims, the invention may be practiced otherwise than as specifically illustrated and described.

What is claimed is:

1. A method for diagnosing systemic inflammatory response in a living organism, comprising:
collecting a first gas sample from the living organism;
measuring the nitrous oxide content of the first gas sample to acquire a first measured nitrous oxide value; and
comparing first measured nitrous oxide value to a nitrous oxide reference value typical for a healthy and similar living organism to determine the presence of systemic inflammatory response.

2. A method for diagnosing sepsis comprising a method according to claim 1.

3. The method of claim 1 wherein the gas sample is either nasally or orally expired.

4. The method of claim 3 wherein the nitrous oxide is endogenously present in the gas sample.

5. The method of claim 1 wherein measuring the nitrous oxide content of the gas sample comprises illuminating the gas sample.

6. The method of claim 5 wherein illuminating the gas sample further comprises passing a light beam from a spectrometer light source through the gas sample.

7. The method of claim 1 further comprising:
collecting a second gas sample from the living organism;
measuring the nitrous oxide content of the second gas sample to acquire a second measured nitrous oxide value; and
comparing the second measured nitrous oxide value to the first measured nitrous oxide value to determine the presence of systemic inflammatory response in the living organism.

8. The method of claim 7 further comprising administering a therapy to treat the living organism before collecting the second gas sample.

9. The method of claim 1 further comprising administering a treatment to the living organism whereby the systemic inflammatory response is inhibited.

10. The method of claim 9 further comprising:
collecting a second gas sample from the living organism;
measuring the nitrous oxide content of the second gas sample to acquire a second measured nitrous oxide value; and
comparing the second measured nitrous oxide value to the first measured nitrous oxide value to determine an effectiveness of the dosage of medication based on a reduced level of nitrous oxide in the second gas sample.

11. The method of claim 1 wherein the living organism comprises a primate.

12. The method of claim 1 wherein measuring the nitrous oxide content of the first gas sample may comprise measuring the content of a nitrous oxide isotope.

13. The method of claim 1 wherein measuring the nitrous oxide content of the first gas sample comprises illuminating the first gas sample with an infrared light.

14. The method of claim 1 wherein the first gas sample further comprises a reference gas and wherein the method further comprises:
measuring the reference gas content of the first gas sample to acquire a measured reference gas value; and
determining a ratio of the first measured nitrous oxide content value to the measured reference gas value to determine the presence or absence of systemic inflammatory response.

15. The method of claim 14 wherein the reference gas comprises carbon dioxide.

16. The method of claim 15 wherein the carbon dioxide is endogenous to the first gas sample collected from tire living organism.

17. A method for diagnosing the presence or absence of an infection in a living organism, the method comprising:
obtaining a first gas sample from the living organism;
measuring at least one biomarker level in the first gas sample to obtain a measured biomarker level, wherein the at least one biomarker comprises nitrous oxide and wherein a measured nitrous oxide level is obtained;
comparing the measured nitrous oxide level with an expected nitrous oxide level for a healthy organism or with a prior measured nitrous oxide level in the living organism; and
diagnosing the presence or absence of foe infection condition based on the comparison.

18. The method of claim 17 wherein the infection condition comprises sepsis.

19. The method of claim 17 wherein the first gas sample is either nasally or orally expired.

20. The method of claim 17 wherein measuring the biomarker level in the first gas sample comprises illuminating the gas sample.

21. The method of claim 20 wherein illuminating the first gas sample further comprises passing a light beam from a spectrometer light source through the first gas sample.

22. The method of claim 17 further comprising:
collecting a second gas sample from the living organism;
measuring the biomarker level of the second gas sample to acquire a second measured biomarker value; and
comparing the second measured biomarker value to the first measured biomarker value to determine the presence of foe infection condition in the living organism.

23. The method of claim 22 further comprising administering a therapy to treat the infection condition of the living organism before collecting the second gas sample.

24. The method of claim 17 further comprising administering a treatment to the living organism whereby the infection condition is inhibited.

25. The method of claim 24 further comprising:
collecting a second gas sample from the living organism;
measuring the biomarker level of the second gas sample to acquire a second measured biomarker value; and
comparing the second measured biomarker value to the first measured biomarker value to determine an effectiveness of the dosage of medication based on a reduced biomarker level in the second gas sample.

26. The method of claim 17 wherein the living organism comprises a primate.

27. The method of claim 17 wherein measuring the biomarker level of the first gas sample may comprise measuring the level of a nitrous oxide isotope.

28. The method of claim 17 wherein measuring the biomarker level of the first gas sample comprises illuminating the first gas sample with an infrared light.

29. The method of claim 17 wherein the first gas sample further comprises a reference gas and wherein the method further comprises:
measuring the reference gas content of the first gas sample to acquire a measured reference gas value; and
determining a ratio of the first measured biomarker content value to the measured reference gas value to determine the presence or absence of the infection condition.

30. The method of claim 29 wherein the reference gas comprises carbon dioxide.

31. The method of claim 30 wherein the carbon dioxide is endogenous to the first gas sample collected from the living organism.

32. A method for diagnosing sepsis in humans comprising:
collecting a first gas sample from a living organism;
measuring a biomarker level present in the first gas sample to acquire a first measured biomarker value;
comparing the first measured biomarker value to biomarker levels for a living organism not having sepsis or for the same living organism at an earlier time to diagnose the presence or absence of sepsis.

33. The method of claim 32 wherein the biomarker comprises nitrous oxide.

34. The method of claim 32 wherein the biomarker comprises a nitrous oxide isotope.

35. The method of claim 32 wherein the gas sample further comprises a reference gas and wherein the method further comprises:
measuring a reference gas level present in the gas sample to acquire a measured reference gas value; and
determining a ratio of the first measured biomarker value to the measured reference gas value to determine the presence or absence of sepsis.

36. The method of claim 35 wherein the reference gas comprises carbon dioxide.

37. The method of claim 36 wherein the carbon dioxide is endogenous to tire first gas sample collected from the living organism.

38. A method of diagnosing systemic inflammatory response in a human comprising: detecting a level of endogenous N₂O in at least one sample of expired air taken from said human, and diagnosing whether said human has systemic inflammatory response based on said level of endogenous N₂O.

39. A system for the analysis of a breath sample, the system comprising:
a means for accepting a gas sample from a living subject;
a means for measuring an amount of endogenous nitrous oxide present in the gas sample; and
a means for analyzing the level of endogenous nitrous oxide in the gas sample to determine the presence or absence of systemic inflammatory response.

40. The system of claim 39 wherein the means for accepting the gas sample further comprises a non-rebreathing valve.

41. The system of claim 39 wherein the means for accepting the gas sample further comprises a face mask to cover a nose and mouth of the living subject.

42. The system of claim 39 wherein the means for measuring comprises a means for illuminating the gas sample.

43. The system of claim 39 wherein the means for measuring comprises an electrochemical cell.

44. The system of claim 39 wherein the means for measuring the level of nitrous oxide present in the gas sample is further adapted to measure a level of reference gas present in the gas sample.

45. The system of claim 44 wherein the reference gas comprises carbon dioxide.

46. The system of claim 44 wherein the means for analyzing nitrous oxide in the gas sample is further adapted to determine a ratio of nitrous oxide to reference gas to determine the presence or absence of systemic inflammatory response.

47. The system of claim 39 wherein the means for accepting the gas sample from the living subject comprises a ventilator.

48. The system of claim 39 wherein the means for accepting the gas sample from the living subject comprises an intubation device.

49. A method for detecting response to therapy in a living organism, the method comprising:
collecting a first gas sample from the living organism;
measuring a nitrous oxide level of the first gas sample to acquire a first measured nitrous oxide value; and
administering a therapy to the living organism;
collecting a second gas sample from the living organism;
measuring a nitrous oxide level of the second gas sample to acquire a second measured nitrous oxide value; and
comparing the first measured nitrous oxide value to the second measured nitrous oxide value to determine a response to the therapy.

50. The method of claim 49 wherein administering a therapy to the living organism comprises giving the organism a dosage of serine protease.

51. The method of claim 49 wherein measuring the nitrous oxide level of the first and second gas samples comprises illuminating both the first and second gas samples.

52. The method of claim 51 wherein illuminating the first gas sample and illuminating the second gas sample comprises passing a light beam from a spectrometer light source through the first gas sample and through the second gas sample.

53. The method of claim 49 further comprising measuring the level of a reference gas present in the first gas sample to determine a first reference gas value and determining a first ratio of nitrous oxide to reference gas based upon the first measured nitrous oxide value and the first reference gas value.

54. The method of claim 53 further comprising measuring the level of a reference gas present in the second gas sample to determine a second reference gas value and determining a second ratio of nitrous oxide to reference gas based upon the second measured nitrous oxide value and the second reference gas value.

55. The method of claim 54 further comprising the first ratio and the second ratio to determine the effectiveness of the therapy.

56. A method for discovering a drug therapy for a living organism, the method comprising:
collecting a first gas sample from the living organism;
measuring tire nitrous oxide level of the first gas sample to acquire a first measured nitrous oxide value; and
administering a therapy to the living organism;
collecting a second gas sample from the living organism; and
measuring the nitrous oxide level of the second gas sample to acquire a second measured nitrous oxide value; and
comparing the first measured nitrous oxide value to the second measured nitrous oxide value to determine an effectiveness of the drug therapy.

57. The method of claim 56 wherein administering a therapy to the living organism comprises giving the organism a dosage of serine protease.

58. The method of claim 56 wherein measuring the nitrous oxide level of the first and second gas samples comprises illuminating both the first and second gas samples.

59. The method of claim 58 wherein illuminating the first gas sample and illuminating the second gas sample comprises passing a light beam from a spectrometer light source through the first gas sample and through the second gas sample.

60. The method of claim 56 further comprising measuring the level of a reference gas present in the first gas sample to determine a first reference gas value and determining a first ratio of nitrous oxide to reference gas based upon the first measured nitrous oxide value and the first reference gas value.

61. The method of claim 60 further comprising measuring the level of a reference gas present in the second gas sample to determine a second reference gas value and determining a second ratio of nitrous oxide to reference gas based upon the second measured nitrous oxide value and the second reference gas value.

62. The method of claim 61 further comprising the first ratio and the second ratio to determine the effectiveness of the drug therapy.
